(19) United States
(12) Patent Application Publication
(10) Pub. No.: US 2014/0335061 A1
(43) Pub. Date: Nov. 13, 2014

(54) SUPPLEMENTED OIL COMPOSITIONS AND METHODS FOR IMPROVED HEALTH

(71) Applicant: Metaproteomics,LLC, Aliso Viejo, CA (US)

(72) Inventors: Matthew L. Tripp, Gig Harbor, WA (US); Robert H. Lerman, Gig Harbor, WA (US)

(73) Assignee: METAPROTEOMICS, LLC, Aliso Viejo, CA (US)

(21) Appl. No.: 14/363,763

(22) PCT Filed: Dec. 7, 2012
(86) PCT No.: PCT/US12/68451

§ 371 Date: Jun. 6, 2014

Related U.S. Application Data
(60) Provisional application No. 61/568,269, filed on Dec. 8, 2011.

(51) Int. Cl. A61K 35/56; A61K 36/02; A61K 31/122; A61K 35/60

(52) U.S. Cl. CPC A61K 35/616; A61K 35/60; A61K 35/61; A61K 35/612; A61K 36/02; A61K 31/122
USPC 424/93.7; 424/523; 424/522; 424/538; 424/195.17

(57) ABSTRACT

Disclosed are compositions and methods comprising supplemented oils for improving health, reducing cardiovascular risk factors, or treating certain conditions.

### CROSS-REFERENCE TO RELATED APPLICATIONS

This patent application claims priority to U.S. provisional application Ser. No. 61/568,269 filed on Dec. 8, 2011.

### BACKGROUND OF THE INVENTION

1. Field of the Invention

The present invention relates generally to compositions and methods comprising supplemented oils for improving health, reducing cardiovascular risk factors, or treating certain conditions.

2. Description of the Related Art

Monocyte activation and adhesion to the endothelium play critical roles in inflammatory and cardiovascular diseases. The process is further complicated by hyperglycemia leading to the complications in diabetes. Both TNFa and hyperglycemia activate many genes involved in the inflammatory responses (Shanmugam N, Gae Gonzalo I T, Natarajan R. Molecular mechanisms of high glucose-induced cyclooxygenase-2 expression in monocytes. Diabetes. 53(3):795-802, 2004.).

MCP-1 is a potent chemoatractant for monocytes and plays a pivotal role in early atherogenesis by promoting monocyte infiltration and adherence to the endothelium, leading to the formation of atherosclerotic plaque (Charo I F, Taubman M B. Chemokines in the pathogenesis of vascular disease. Circ Res. 29; 95(9):858-66, 2004.).

Matrix metalloproteinases (MMPs) are the primary proteolytic enzymes in the extracellular space, contributing to weakening of the plaque cap via their ability to cleave the extracellular matrix (ECM) (Nagase H, Visse R, Murphy G. Structure and function of matrix metalloproteinases and TIMPs. Cardiovasc Res 2006; 69:562-73.). Atherosclerotic plaque rupture, causally related to the majority of acute coronary syndromes, commonly occurs at sites of continuous inflammation and collagen degradation (Virmani R, Kolodgie F D, Burke A P. Farb A, Schwartz S M. Lessons from sudden coronary death: a comprehensive morphological classification scheme for atherosclerotic lesions. Arterioscler Thromb Vasc Biol 2000; 20:1262-75.).

Clinical and experimental studies have implicated MMP-9 (gelatinase B) as a key determinant of atherosclerotic plaque stability (Gough P J, Gomez I G, Wille P T, Raines E W. Macrophage expression of active MMP-9 induces acute plaque disruption in apoE-deficient mice. J Clin Invest 2006; 116:59-69.; Fukuda D, Shimada K, Tanaka A, Kusuyama T. Yamashita H, Ehara S, et al. Comparison of levels of serum matrix metalloproteinase-9 in patients with acute myocardial infarction versus unstable angina pectoris versus stable angina pectoris. Am J Cardiol 2006; 97:175-80.; Blankenberg S, Rupprecht H J, Poirier O, Bickel C. Smieja M, Hafner G, et al. Plasma concentrations and genetic variation of matrix metalloproteinase 9 and prognosis of patients with cardiovascular disease. Circulation 2003; 107:1579-85.). MMP-9 principally derives from monocytes/macrophages (Chase A J, Bond M, Crook M E, Newby A C. Role of nuclear factor kappa B activation in metalloproteinase-1, -3, and -9 secretion by human macrophages in vitro and rabbit foam cells produced in vivo. Arterioscler Thromb Vase Biol 2002; 22:765-71.; Stawowy P, Meyborg H, Stibenz D, Borges Pereira Stawowy N, Roser M, Thanabalasingam U, et al. Furin-like proprotein convertases are central regulators of the membrane type matrix metalloproteinase-promatrix metalloproteinase-2 proteolytic cascade in atherosclerosis. Circulation 2005; 111:2820-7.), the major cell type involved in the initiation, progression and complications of atherosclerosis. In MNCs MMP-9 is strongly inducible by a number of inflammatory mediators, including TNF-α (Stawowy P, Meyborg Stibenz D. Borges Pereira Stawowy N, Roser M, Thanabalasingam U. et al. Furin-like proprotein convertases are central regulators of the membrane type matrix metalloproteinase-promatrix metalloproteinase-2 proteolytic cascade in atherosclerosis. Circulation 2005; 111:2820-7.).

It has previously been shown that the anti-inflammatory compounds such as aspirin, glucocorticoids, and curcumin exert their effects through the inhibition of NE-κB signaling pathways (Kopp E and Ghosh S. Inhibition of NF-kappa B by sodium salicylate and aspirin. Science, 22: 270 (5244): 2017-9,1994.; De Bosscher K, Schmitz M L, Vanden Berghe W, Plaisance S, Fiers W, Haegeman G. Glucocorticoid-mediated repression of nuclear factor-kappaB-dependent transcription involves direct interference with transactivation. Proc Natl Acad Sci USA. 9; 94(25):13504-9, 1997.; Pan M H, Lin-Shiau S Y, Ho C T, Lin J H, Lin J K. Suppression of lipopolysaccharide-induced nuclear factor-kappaB activity by theaflavin-3,3′-digallate from black tea and other polyphenols through down-regulation of IkappaB kinase activity in macrophages. Biochem Pharmacol. 15; 59(4):357-67, 2000.).

Inflammatory mediators, such as TNFa activate NFkB, which regulate the expression of many genes involved in the inflammatory responses such as proinflammatory cytokines, adhesion molecules, chemokines including monocyte chemotactic protein-1 (MCP-1) (Ueda A, Ishigatsubo Y, Okubo T, Yoshimura T. Transcriptional regulation of the human monocyte chemoattractant protein-1 gene. Cooperation of two NF-kappaB sites and NF-kappaB/Rel subunit specificity. J Biol Chem. 5; 272(49):31092-9, 1997.). It has been shown that hyperglycemia activate inflammation through the activation of PKC and NF-kB signaling pathways in monocytes (Devaraj S, Venugopal S K, Singh U, Jialal I. Hyperglycemia induces monocytic release of interleukin-6 via induction of protein kinase c-{alpha} and -{beta}.Diabetes.; 54(1):85-91, 2005.; Shanmugam N, Gae Gonzalo I T, Natarajan R. Molecular mechanisms of high glucose-induced cyclooxygenase-2 expression in monocytes. Diabetes. 53(3):795-802, 2004.)

Endothelial derived Nitric Oxide (NO) is a key determinant of cardiovascular homeostasis modulating vascular endothelial responsiveness and thus regulating systemic blood pressure, vascular remodeling and angiogenesis (Moncada S, Higgs A: The L-Arginine-Nitric Oxide pathway. NEJM 1993; 329:2002-12). An important stimulus for the continuous production of NO is viscous drag related to blood flow across the endothelium. Endothelial NO synthase (eNOS) is under direct regulation by the protein kinase Akt. Shear stress and hyperglycemia through a series of mediating kinases directly activation Akt. (Dimmeler S, Fleming I, Fisslthaler B, Hermann C, Busse R, Zeiher A M. Activation of nitric oxide synthase in endothelial cells by Akt-dependent phosphorylation. Nature 1999; 399:601-5). One of these kinases which actually inhibit Akt is PKCβ. PKCβ is activated by hyperglycemia. Hyperglycemia has been directly shown to inhibit endothelial dependent vasodilation (Rammos G, Peppes V, Zakopoulos N. Transient Insulin Resistance in Normal Subjects: Acute Hyperglycemia inhibits Endothelial-Dependent Vasodilation in Normal Subjects. Metabolic Syndrome and Related Disorders 2008; 6(3):159-170.). Roboxistaurin inhibits PKCβ and thus normalizes endothelial function as measured by Flow Mediated Vasodilation (FMD) (Mehta N N, Sheetz M, Price K, Comiskey L, Amrutia S, Iqbal N, Mohler E R, Reilly M P. Selective PKC beta inhibition with roboxistaurin and endothelial function in type-2 diabetes mellitus. Cardiovasc Drugs Ther 2009; 23(1):17-24). FMD is a non-invasive ultrasonographic technique for measuring brachial artery flow physiology after an induced hypoxemia (Corretti M C et al. Guidelines for the Ultrasound Assessment of Endothelial-Dependent Flow-Mediated Vasodilation of the Brachial Artery—A Report of the International Brachial Artery Reactive Task Force. J Am Coll Cardiol 2002; 39(2): 257-65).

The inventors have discovered that the health benefits of dietary intake of certain naturally occurring plant and animal oils, marine oil concentrates for example, may be improved upon by supplementation of the oil with naturally occurring or derived compounds or ingredients.

### SUMMARY OF THE INVENTION

The present invention relates generally to compositions and methods comprising supplemented oils for improving health, reducing cardiovascular risk factors, or treating certain conditions.

A first embodiment of the invention describes a supplemented oil composition comprising therapeutically effective amounts of a first component being a marine oil concentrate and a second component being a compound selected from the group consisting of resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin. Under this embodiment the composition is in a dosage form suitable for oral administration where the composition comprises from at least 30 wt % to about 75 wt % of w-3 long chain polyunsaturated fatty acids selected from DHA and EPA and the DHA:EPA ratio is from 10:0.01 to 1:10. The therapeutically effective amounts are for use in treating a condition selected from the group consisting of mild to moderate hypertriglyceridemia, diabetes, atherosclerosis, mental cognition, metabolic syndrome, inflammation, and inflammation related disorders.

Another embodiment of the invention describes methods of using a supplemented oil composition comprising therapeutically effective amounts of a first component being a marine oil concentrate and a second component being a compound selected from the group consisting of resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin. Under this embodiment the composition is in a dosage form suitable for oral administration where the composition comprises from at least 30 wt % to about 75 wt % of w-3 long chain polyunsaturated fatty acids selected from DHA and EPA and the DHA:EPA ratio is from 10:0.01 to 1:10. The therapeutically effective amounts are for use in treating a condition selected from the group consisting of mild to moderate hypertriglyceridemia, diabetes, atherosclerosis, mental cognition, metabolic syndrome, inflammation, and inflammation related disorders.

### BRIEF DESCRIPTION OF THE DRAWINGS

FIG. 1. Percent changes in flow-mediated vasodilation (FMD) after a 12-week treatment with THIAA/niacin or placebo. A, data from 11 subjects completed the study (N=7 for THIAA/niacin arm and N=4 for placebo). B, data from the optional follow-up study in which 2 placebo arm participants (after completing the trial) volunteered to receive THIAA/niacin treatment for 10-12 weeks and their data were combined with the original THIAA/niacin arm (N=9). Placebo arm data remained unchanged (N=4).

FIG. 2. A graphic representation of the omega-3 index measured at baseline and at the end of the trial for a multi-center randomized trial conducted in which women with metabolic syndrome (N=58) who consumed an MLG diet for 12 weeks. Panel A shows that the MLG diet increased the omega-3 index by 14.9%, from 4.35% to 5.00% (P<0.001). In panel B, it is shown that meanwhile, RBC trans fatty acid content decreased by 8.9%, from 1.24% to 1.13% (P<0.001).

FIG. 3. A graphic representation of the omega-3 index measured at baseline and at the end of the trial for a case series study in which women with metabolic syndrome (N-12) were give, in addition to the MLG diets, one of two omega-3 supplements for 12 weeks. Panel A shows that in the 7 women who received Supplement A (High DHA™, containing 600 mg DHA+60 mg EPA per softgel), 1 gel 3 times/day, the omega-3 index increased by 92.5%, from 3.97% to 7.72% (P<0.001). Panel B shows that in the 5 women who received Supplement B (EPA-DHA 720™, containing 580 mg DHA+860 mg EPA per softgels), 2 gels twice/day, the omega-3 index increased by 85.4%, from 4.85% to 8.88% (P<0.001).

FIG. 4. Graphic representation of the effects of THIAA (tetrahydroisoalpha acids) alone or in conjunction with a marine oil concentrate on serum triglyceride (TG) levels.

### DETAILED DESCRIPTION OF THE INVENTION

The present invention relates generally to compositions and methods comprising supplemented oils for improving health, reducing cardiovascular risk factors, or treating certain conditions.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables that are inherently discrete, and can be 0.0, 0.1, 0.01, 0.001, or any other real value for variables that are inherently continuous.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

A first embodiment of the invention describes a supplemented oil composition comprising therapeutically effective amounts of a first component being a marine oil concentrate and a second component being a compound selected from the group consisting of resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin. Under this embodiment the composition is in a dosage form suitable for oral administration where the composition comprises from at least 30 wt % to about 75 wt % of w-3 long chain polyunsaturated fatty acids selected from DHA and EPA and the DHA:EPA ratio is from 10:0.01 to 1:10. The therapeutically effective amounts are for use in treating a condition selected from the group consisting of mild to moderate hypertriglyceridemia, diabetes, atherosclerosis, mental cognition, metabolic syndrome, inflammation, and inflammation related disorders.

In an aspect of this embodiment, the dosage form provides from 250 mg to 1,250 mg of the marine oil concentrate.

In another aspect, in the composition modulates one or more cardiovascular risk factors selected from the group consisting of omega-3 index, total cholesterol, LDL-cholesterol (direct measurement), HDL-cholesterol, Apo B, ApoA-1, Hs-CRP, albumin, ALAT, Alk Phosphatase, bilirubin, INR, TSH, BUN/Creatinine, complete blood count (CBC), aldosterone, angiotensin 2, oxidized LDL, resolvins, and renin.

As used herein, a “supplemented oil composition” refers to a naturally occur plant or animal derived oil, either concentrated or non-concentrated, which has had compounds or ingredient added which do not occur naturally in the oil. Examples of compounds or ingredients which may be used to supplement include, without limitation resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin.

“CoQ10” as used refers to coenzyme Q10 or ubiquinone. CoQ10 may be provided either in a natural state or as a synthetic analog. As used herein, “ECGC” refers to epigallocatechin gallate, also known as epigallocatechin 3-gallate.

A “marine oil concentrate” refers to an oil composition where the omega-3 fatty acids eicosapentaenoic acid (EPA), and docosahexaenoic acid (DHA) are concentrated to levels above that occurring naturally. Representative marine oil sources include, without limitation a member selected from the group consisting of fish, squid, micro algae, macro algae, krill, sea cucumber, skates, rays, sharks, and crustaceans.

As used herein, “tetrahydro-isoalpha acid” or “THIAA” refers to any of one or more of tetrahydro-isoadhumulone, tetrahydro-isocohumulone and tetrahydro-isohumulone. Structurally, THIAA comprises compounds of Genus A

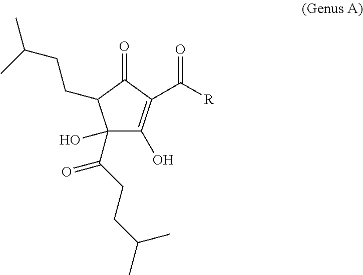

wherein tetrahydro-isohumulone (R=—CH₂CH(CH₃)₂); tehydro-isocohumulone (R=CH(CH₃)₂); and tetrahydro-isoadhumulone (R=—CH(CH₃)(CH₂CH₃)) are shown.

The term “acacia”, as used herein, refers to any member of leguminous trees and shrubs of the genus Acacia. Preferably, the botanical compound derived from acacia is derived from Acacia catechu or Acacia nilotica. In those aspects where the acacia derived compound or extract is derived from Acacia catechu or Acacia nilotica, the Acacia catechu or Acacia nilotica compound is selected from the group consisting of gum resin, bark powder, heartwood powder, and an Acacia catechu or Acacia nilotica extract. In those aspects where the acacia derived compound is an Acacia catechu or Acacia nilotica extract, the extract is selected from acidic, alkaline, polar solvent, nonpolar solvent, and gastric fluid extracts.

As used herein, dihydro-isoalpha acids refers to those compounds generally described as reduced isoalpha acids commonly associated with hops and beer production, more specifically dihydro-isoalpha acids refers to compounds of Genus B

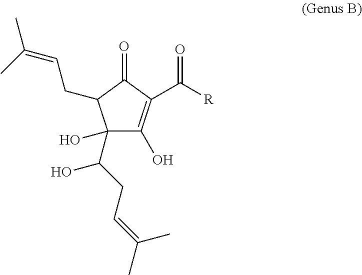

wherein dihydro-isohumulone (R=—CH₂CH(CH₃)2); dihydro-isocohumulone (R=—CH₃)₂); and dihydro-isoadhumulone (R=—CH(CH₃)CH₂CH₃) are shown. “Rho” refers to those reduced isoalpha acids wherein the reduction is a reduction of the carbonyl group in the 4-methyl-3-pentenoyl side chain.

As used herein, “RIAA” refers to any mixture of one or more of dihyro-isoadhumulone, dihydro-isocohumulone and dihydro-isohumulone.

As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc.

It will be understood that the subject to which a compound of the invention is administered need not suffer from a specific traumatic state. Indeed, the compounds of the invention may be administered prophylactically, prior to any development of symptoms. The term “therapeutic.” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

It will be appreciated by those of skill in the art that the number of administrations of the compounds according to the invention will vary from patient to patient based on the particular medical status of that patient at any given time including other clinical factors such as age, weight and condition of the mammal and the route of administration chosen.

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

Compounds according to the invention may be present as salts. In particular, pharmaceutically acceptable salts of the compounds are contemplated. A “pharmaceutically acceptable salt” of the invention is a combination of a compound of the invention and either an acid or a base that forms a salt (such as, for example, the magnesium salt, denoted herein as “Mg” or “Mag”) with the compound and is tolerated by a subject under therapeutic conditions. In general, a pharmaceutically acceptable salt of a compound of the invention will have a therapeutic index (the ratio of the lowest toxic dose to the lowest therapeutically effective dose) of 1 or greater. The person skilled in the art will recognize that the lowest therapeutically effective dose will vary from subject to subject and from indication to indication, and will thus adjust accordingly.

The compounds according to the invention are optionally formulated in a pharmaceutically acceptable vehicle with any of the well known pharmaceutically acceptable carriers, including diluents and excipients [see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995]. While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well as any other pharmacologically active ingredient useful for the treatment of the symptom/condition being treated.

The compounds of the present invention may be provided in a pharmaceutically acceptable vehicle using formulation methods known to those of ordinary skill in the art. The compositions of the invention can be administered by standard routes, though preferably administration is by inhalation routes. The compositions of the invention include those suitable for oral, inhalation, rectal, ophthalmic (including intravitreal or intracameral), nasal, topical (including buccal and sublingual), vaginal, or parenteral (including subcutaneous, intramuscular, intravenous, intradermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powders and aerosols. The present invention encompasses liquid and powdered compositions suitable for nebulization and intrabronchial use, or aerosol compositions administered via an aerosol unit dispensing metered doses (“MDI”). The active ingredient may be formulated in an aqueous pharmaceutically acceptable inhalant vehicle, such as, for example, isotonic saline or bacteriostatic water and other types of vehicles that are well known in the art. The solutions are administered by means of a pump or squeeze-actuated nebulized spray dispenser, or by any other conventional means for causing or enabling the requisite dosage amount of the liquid composition to be inhaled into the patient's lungs. Powder compositions containing the anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule which may be inserted by the patient into a device that punctures the capsule and blows the powder out in a steady stream. Aerosol formulations for use in the subject method typically include propellants, surfactants, and co-solvents and may be filled into conventional aerosol containers that are closed by a suitable metering valve.

Formulations of compositions of the present invention suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention.

For oral administration, the compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

Formulations of compositions according to the aspects of the present invention suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein.

Formulations of compositions of the present invention for rectal administration may be prepared as a suppository with a suitable base comprising, such as, for example, cocoa butter.

Formulations of compositions of the present invention suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier. Formulations of compositions of the present invention suitable for topical administration to the skin may be presented as ointments, creams, gels, lotions and pastes comprising the ingredient to be administered in a pharmaceutical acceptable carrier. A topical delivery system contemplated is a transdermal patch containing the ingredient to be administered.

Formulations of compositions according to the aspects of the present invention suitable for vaginal administration may be presented as pessaries, suppositories, tampons, creams, gels, pastes, foams or spray formulations containing in addition to the compound of the invention such pharmaceutically acceptable carriers as are known in the art to be appropriate.

The methods and compositions of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

As used herein, “metabolic syndrome” and “diabetes associated disorders” refers to insulin related disorders, i.e., to those diseases or conditions where the response to insulin is either causative of the disease or has been implicated in the progression or suppression of the disease or condition. Representative examples of insulin related disorders include, without limitation diabetes, diabetic complications, insulin sensitivity, polycystic ovary disease, hyperglycemia, dyslipidemia, insulin resistance, metabolic syndrome, obesity, body weight gain, inflammatory diseases, diseases of the digestive organs, stenocardia, myocardial infarction, sequelae of stenocardia or myocardial infarction, senile dementia, and cerebrovascular dementia. See, Harrison's Principles of Internal Medicine, 16h Ed., McGraw Hill Companies Inc., New York (2005).

“Insulin resistance” refers to a reduced sensitivity to insulin by the body's insulin-dependent processes resulting in lowered activity of these processes or an increase in insulin production or both. Insulin resistance is typical of type 2 diabetes but may also occur in the absence of diabetes.

As used herein “diabetic complications” include, without limitation, retinopathy, muscle infarction, idiopathic skeletal hyperostosis and bone loss, foot ulcers, neuropathy, arteriosclerosis, respiratory autonomic neuropathy and structural derangement of the thorax and lung parenchyma, left ventricular hypertrophy, cardiovascular morbidity, progressive loss of kidney function, and anemia.

“Inflammation” or “inflammatory condition” as used herein refers to a local response to cellular injury that is marked by capillary dilatation, leukocytic infiltration, redness, heat, pain, swelling, and often loss of function and that serves as a mechanism initiating the elimination of noxious agents and of damaged tissue. Representative symptoms of inflammation or an inflammatory condition include, if confined to a joint, redness, swollen joint that's warm to touch, joint pain and stiffness, and loss of joint function. Systemic inflammatory responses can produce “flu-like” symptoms, such as, for instance, fever, chills, fatigue/loss of energy, headaches, loss of appetite, and muscle stiffness.

Examples, without limitation, of inflammatory conditions include diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

As used herein, “mild to moderate hypertriglyceridemia” refers to individuals having a serum triglyceride level between 150 and 450 mg/dL.

Another embodiment of the invention describes methods of using a supplemented oil composition comprising therapeutically effective amounts of a first component being a marine oil concentrate and a second component being a compound selected from the group consisting of resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin. Under this embodiment the composition is in a dosage form suitable for oral administration where the composition comprises from at least 30 wt % to about 75 wt % of w-3 long chain polyunsaturated fatty acids selected from DHA and EPA and the DHA:EPA ratio is from 10:0.01 to 1:10. The therapeutically effective amounts are for use in treating a condition selected from the group consisting of mild to moderate hypertriglyceridemia, diabetes, atherosclerosis, mental cognition, metabolic syndrome, inflammation, and inflammation related disorders.

In an aspect of this embodiment, the dosage form provides from 250 mg to 1,250 mg of the marine oil concentrate.

In another aspect, in the composition modulates one or more cardiovascular risk factors selected from the group consisting of omega-3 index, total cholesterol, LDL-cholesterol (direct measurement), HDL-cholesterol, Apo B, ApoA-1, Hs-CRP, albumin, ALAT, Alk Phosphatase, bilirubin, INR, TSH, BUN/Creatinine, complete blood count (CBC), aldosterone, angiotensin 2, oxidized LDL, resolvins, and renin.

In some aspects of this embodiment, the daily dose of the marine oil concentrate is from 1 gm to 6 gm.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

### EXAMPLES

### Example 1

### Effect of THIAA/Niacin on Flow Mediated Dilation

To investigate the effect of THIAA/niacin on FMD (flow mediated dilation) in 11 volunteers with dyslipidemia, a randomized, placebo-controlled trial was conducted at the Functional Medicine Research Center (Gig Harbor, Wash.). Inclusion criteria included (1) age between 30 and 60 years old, (2) LDL-C≧130 mg/dL, (3) HDL-C≦50 mg/dL for men or ≦60 mg/dL for women, and (4) willingness to maintain current dietary and exercise practice during the study. Key exclusion criteria included (1) use of dietary supplements that contained the active ingredient(s) in this study with the exception of multi-vitamins that contain no more than 25 mg niacin, (2) use of NSAIDs and COX-2 inhibitors in the preceding 2 weeks and oral corticosteroids in the preceding 4 weeks, (3) subjects who were ≧300 lbs. (4) subjects with a history of coronary artery disease, arrhythmia, cerebrovascular accident, HIV infection, cancer,

or significant liver or kidney disease, and (5) pregnant and nursing women. The study was conducted in accordance with the Declaration of Helsinki, and informed written consent was obtained from each participant prior to enrollment in the trial.

At the beginning of the trial, eligible participants provided fasting blood samples, and had their brachial FMD measured by the study doctor. Participants were then randomized to receive either the placebo tablet (N=4) or the THIAA/niacin combined tablet (N-7; 1 tablet twice daily with food) for 12 weeks, during which they were instructed to maintain their normal dietary and exercise practice. Participants returned to the clinic every 2 weeks for the evaluation of compliance and potential adverse events. At the end of the 12 weeks, participants provided fasting blood samples and had their FMD measured by the same study doctor.

Blood samples collected at baseline and at end of trial were sent to Quest Diagnostics (Seattle, Wash.) for analyses of lipids, glucose, high-sensitivity C-reactive protein (hs-CRP), and uric acid. The FMD measurements were performed on a MicroMaxx® Ultrasound System (SonoSite, Bothell, Wash.) using the protocol following the guidelines reported by Corretti et al. [Corretti M C, Anderson T J, Benjamin E J, et al., Guidelines for the ultrasound assessment of endothelial-dependent flow-mediated vasodilation of the brachial artery: a report of the International Brachial Artery Reactivity Task Force. J Am Coll Cardiol, 2002. 39(2): p. 257-265.]. Participants were instructed to fast for 12 hours and minimize their physical activities, and rest for 15 minutes in supine position prior to the procedure. The participant's blood pressure was monitored every 3-5 minutes for safety purposes utilizing an automated sphygmomanometer with the blood pressure cuff placed around the participant's left arm. An automatic inflation, narrow-width occlusion cuff was place as high as possible on the participant's right arm. The brachial artery of the right arm approximately 5-10 cm above the antecubital fossa was scanned with a 10.5 MHz operating frequency. The cuff was then inflated to 40 mm Hg above the participant's systolic pressure or at least 200 mm Hg for initial occlusion of the brachial artery. After 5 minutes of occlusion, the pressure in the cuff was rapidly released and post-stimulus image acquisition was recorded until 3 minutes after the cuff release. The relative increase in diameter compared to the baseline diameter was calculated (as percentage) using Vascular Research Tools version 5.7.6 (Medical Imaging Associates, LLC., Coralville, Iowa).

For in vitro data, repeated measures ANOVA was applied to analyze the effects of THIAA, niacin and THIAA/niacin. Data were analyzed using GraphPad Prism software (San Diego, Calif.) and reported as mean ±SD. For the pilot clinical study data, two-sample t-tests were performed to compare biomarkers between THIAA/niacin arm and placebo arm at baseline and at end of trial. T-tests were also utilized for comparing changes from baseline between arms. For the within-arm changes from baseline, paired t-tests were conducted to determine the significance. The probability of a type I error was set at the nominal 5 percent level. Data were analyzed using SAS (software version 8.1, SAS Institute) and reported as means ±SE.

Results: The averaged age in the THIAA/niacin arm, including 3 men and 4 women. was 49.7±8.2 years old (mean±SD). Their baseline BMI was 33.1±5.4 kg/m². The average age in the placebo arm (1 man and 3 women) was 56.1±1/3 years old and their BMI was 33.0±7.7 kg/m². Baseline serum lipids, glucose, hs-CRP, and uric acid did not differ between study arms. In the THIAA/niacin arm, there was a significant decrease (P<0.05) in total cholesterol, LDL-C and uric acid at week 12 compared to baseline (Table 1). A trend toward a decrease in apolipoprotein B (apo B) (P=0.07). In contrast, no differences in all measurements were observed in the placebo arm except there was a significant increase in hs-CRP (P=0.03) at week 12. A significant between-arm difference (P<0.05) was seen in hs-CRP and glucose.

TABLE 1
Values and changes of lipid markers, hs-CRP, glucose, and uric acid by study arms.

| | | THIAA/niacin arm | | Placebo arm | | |
|---|---|---|---|---|---|---|
| Variable | Visit | Value | Change | Value | Change | P |
| Cholesterol | Baseline | 236.1 ± 8.0 | | 248.3 ± 18.8 | | |
| (mg/dL) | 12 weeks | 219.1 ± 5.9 | −17.0 ± 6.5* | 246.0 ± 23.8 | −2.3 ± 9.3 | 0.22 |
| TG | Baseline | 215.6 ± 59.1 | | 185.3 ± 31.5 | | |
| (mg/dL) | 12 weeks | 186.9 ± 37.0 | −28.7 ± 27.4 | 212.3 ± 61.4 | 27.0 ± 38.4 | 0.26 |
| LDL | Baseline | 152.9 ± 8.7 | | 160.5 ± 17.7 | | |
| (mg/dL) | 12 weeks | 133.7 ± 6.6 | −19.1 ± 6.1* | 153.3 ± 14.3 | −7.3 ± 4.3 | 0.21 |
| HDL | Baseline | 47.9 ± 2.7 | | 50.5 ± 1.6 | | |
| (mg/dL) | 12 weeks | 48.1 ± 4.7 | 0.3 ± 2.5 | 50.3 ± 3.4 | −0.3 ± 4.4 | 0.91 |
| Chol/HDL | Baseline | 5.00 ± 0.23 | | 4.98 ± 0.50 | | |
| | 12 weeks | 4.83 ± 0.54 | −0.17 ± 0.34 | 4.98 ± 0.64 | 0.00 ± 0.35 | 0.75 |
| TG/HDL | Baseline | 4.95 ± 1.69 | | 3.73 ± 0.71 | | |
| | 12 weeks | 4.59 ± 1.45 | −0.36 ± 0.37 | 4.29 ± 1.35 | 0.56 ± 0.97 | 0.31 |
| Apo A1 | Baseline | 142.3 ± 2.6 | | 146.3 ± 3.2 | | |
| (mg/dL) | 12 weeks | 135.6 ± 9.9 | −6.7 ± 8.0 | 143.3 ± 4.5 | −3.0 ± 2.6 | 0.67 |
| Apo B | Baseline | 112.6 ± 4.2 | | 131.3 ± 12.5 | | |
| (mg/dL) | 12 weeks | 110.3 ± 5.6 | −12.3 ± 5.6 | 125.3 ± 9.5 | −6.0 ± 4.7 | 0.47 |
| ApoB/ApoA1 | Baseline | 0.86 ± 0.02 | | 0.89 ± 0.07 | | |
| | 12 weeks | 0.83 ± 0.06 | −0.03 ± 0.06 | 0.87 ± 0.07 | −0.02 ± 0.04 | 0.92 |
| Lp(a) | Baseline | 108.3 ± 30.5 | | 95.5 ± 72.3 | | |
| (nmol/L) | 12 weeks | 99.0 ± 30.4 | −9.3 ± 8.3 | 94.5 ± 72.6 | −1.0 ± 1.3 | 0.36 |
| hs-CRP | Baseline | 3.5 ± 1.0 | | 5.0 ± 2.0 | | |
| (mg/L) | 12 weeks | 3.3 ± 1.0 | −0.3 ± 0.2 | 5.7 ± 2.1 | 0.7 ± 0.2* | 0.03 |
| Glucose | Baseline | 105.6 ± 12.5 | | 100.8 ± 4.7 | | |
| (mg/dL) | 12 weeks | 108.9 ± 11.9 | 3.3 ± 1.6 | 96.5 ± 2.4 | −4.3 ± 3.5 | 0.05 |
| Uric acid | Baseline | 6.6 ± 0.7 | | 7.6 ± 0.9 | | |
| (mg/dL) | 12 weeks | 6.1 ± 0.5 | −0.6 ± 0.2* | 7.6 ± 0.9 | 0.0 ± 0.1 | 0.11 |

*P < 0.05

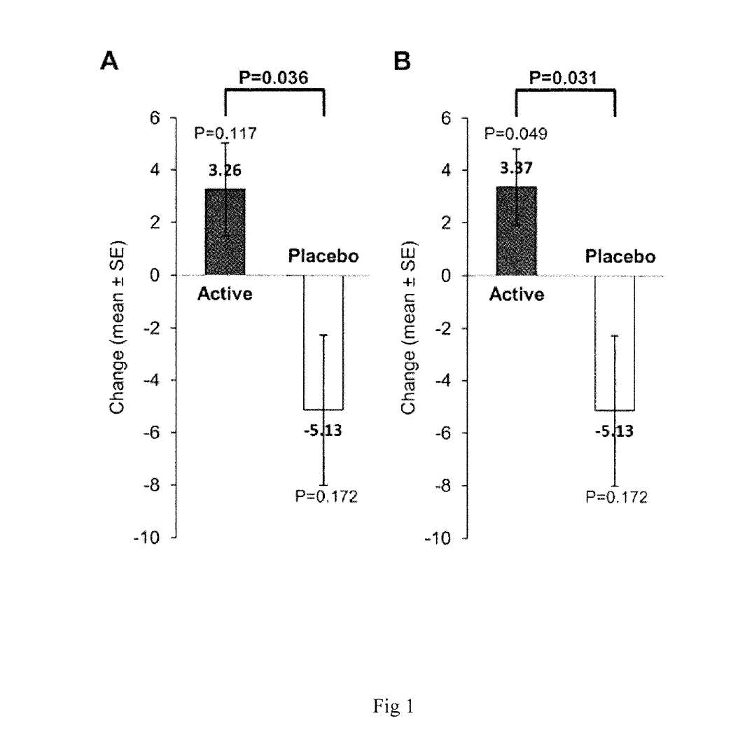

Baseline FMD measurements did not differ between arms. By the end of week 12, a trend toward an improvement was observed in the THIAA/niacin arm whereas there was a trend toward a decrease in the placebo arm; the difference between arms was statistically significant (P=0.036, FIG. 1A). In the optional follow-up study in which 2 placebo arm participants (after completing the trial) volunteered to receive THIAA/niacin treatment for 10-12 weeks, the combined data (7 data points from the original THIAA/niacin arm plus the 2 new data points) showed a statistically significant improvement in FMD (P=0.049, FIG. 1B) compared to baseline. The between-arm difference was also statistically significant (P=0.031).

Fourteen adverse events, occurring in 7 subjects, were noted during the study. Events were mild to moderate in severity and were deemed to have no or unlikely causality. During study product consumption, 2 respiratory infections, mild headaches, one accidental fall, and back discomfort were noted. THIAA/niacin did not differ from placebo in regards to physical symptoms and reported adverse events.

### Example 1

### Correction of the Omega-3 Index in Women With Metabolic Syndrome

Objective: To determine the effects a Mediterranean style low-glycemic-load diet (MLG) with or without omega-3 fatty acid supplementation on the omega-3 index in women with metabolic syndrome.

Calculation of the Omega-3 Index: The omega-3 index was calculated based on the formula:

$$Omega \text{-} 3 index = \frac{EPA + DHA in RBC}{Total fatty acids in RBC} \times 100$$

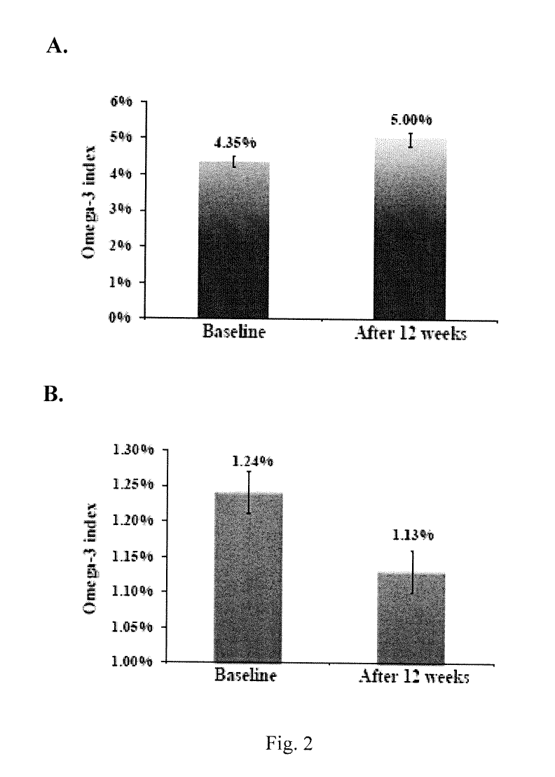

Study 1: A multi-center randomized trial was conducted in which women with metabolic syndrome (N=58) consumed an MLG diet for 12 weeks. The omega-3 index was measured at baseline and at the end of the trial. The results are presented in FIG. 2.

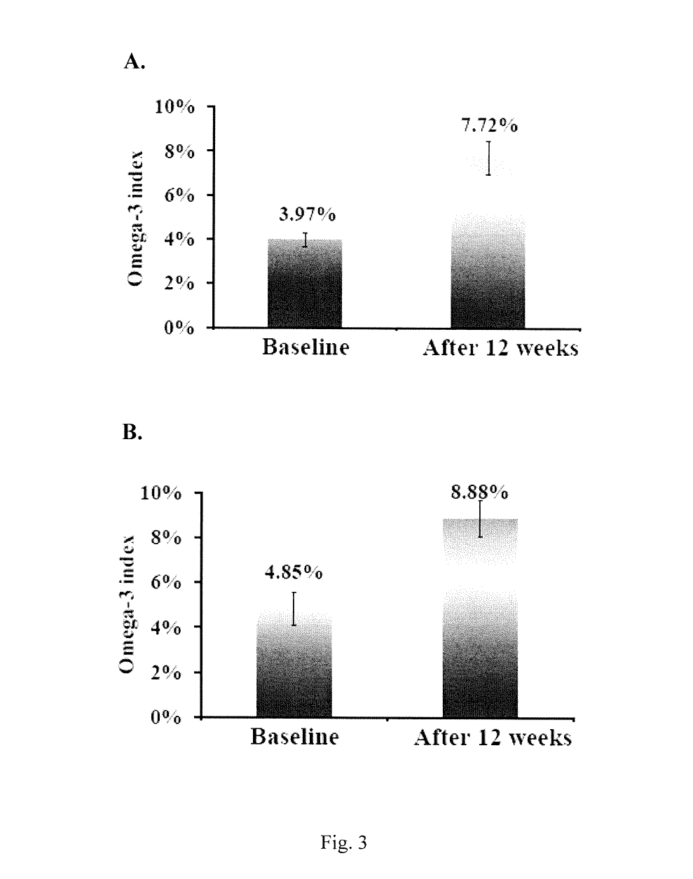

Study 2: A case series study in which women with metabolic syndrome (N=12) were give, in addition to the MLG dies, one of two omega-3 supplements for 12 weeks. The omega-3 index was measured at baseline and at the end of the trial. The results are presented in FIG. 3.

### Example 2

### Triglyceride Reduction Using THIAA

Objective: To determine the effects of THIAA (tetrahydroisoalpha acids) on serum triglyceride (TG) levels in volunteers.

Study 1: In this study, twelve (12) volunteers with a fasting serum glucose level were enrolled and given THIAA (400 mg/dose; 3×/day). Serum triglyceride levels were measured at study initiation and at week 4 and 8 of the study. Volunteers were instructed to maintain their baseline diet and exercise levels and medications. Ten (10) volunteers completed the study.

Results: The results, presented in Table 2 below, show that four weeks of THIAA treatment resulted in an average reduction of serum TG levels of 5.5% from baseline and a 2.6% reduction in TG levels after eight weeks.

TABLE 2
Effect of THIAA on Triglyceride Levels

| | TG | | |
|---|---|---|---|
| | Baseline | 4 weeks | 8 weeks |
| | 195 | 208 | 183 |
| | 185 | 97 | 137 |
| | 232 | 174 | 128 |
| | 267 | 206 | 190 |
| | 295 | 389 | 292 |
| | 261 | 144 | 166 |
| | 445 | 452 | 525 |
| | 219 | 235 | 194 |
| | 295 | 376 | 515 |
| | 274 | 239 | 268 |
| Mean | 266.8 | 252 | 259.8 |
| S.D. | 73.61582 | 115.6758 | 146.4057 |

Study2: In this study, eight (8) volunteers with a fasting serum glucose level were enrolled and given 1350 mg of a marine oil concentrate containing 375 mg each of EPA and DHA (3×/day)for the first 4 weeks with the addition of THIAA (400 mg/dose; 3×/day) for 8 additional weeks. Serum triglyceride levels were measured at study initiation and at week 4 and 8 of the study. Volunteers were instructed to maintain their baseline diet and exercise levels and medications. Seven (7) volunteers completed the study.

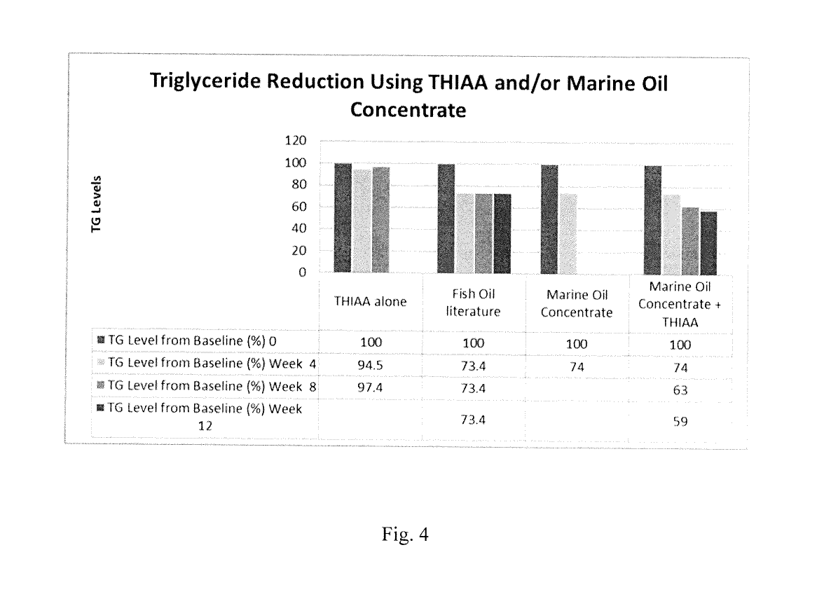

Results: The results, presented in Table 3 below, show that four weeks of marine oil concentrate alone resulted in a 26% reduction in TG levels (comparable to that seen in the literature for 8 weeks of fish oil treatment alone in humans. (see Oelrich B et al, Effect of fish oil supplementation on serum triglycerides, LDL cholesterol and LDL subfractions in hypertriglyceridemic adults., Nutr Metab Cardiovasc Dis. 2011 Sep 15. [Epub ahead of print]; Becker D J, et al, Simvastatin vs therapeutic lifestyle changes and supplements: randomized primary prevention trial., Mayo Clin Proc. 2008 Jul; 83(7):758-64.; Swahn E et al. Omega-3 Ethyl Ester Concentrate Decreases Total Apolipoprotein CIII and Increases Antithrombin III in Postmyocardial Infarction Patients., Clin Drug Investig. 1998; 15(6):473-82.). Further supplementation with THIAA resulted in a 37% and 41% reduction in TG levels at weeks 8 and 12 respectively. Such results are considered supradditive and are presented in Table 3 below and FIG. 4.

TABLE 3
Effect of a THIAA Supplemented Marine
Oil Concentrate on Triglyceride Levels

| | TG | | | |
|---|---|---|---|---|
| | Baseline | EP750 | EP750 + THIAA | EP750 + THIAA |
| | 437 | 264 | 185 | 205 |
| | 226 | 146 | 135 | 151 |
| | 176 | 207 | 155 | 150 |
| | 275 | 321 | 259 | 216 |
| | 495 | 292 | 322 | 245 |
| | 327 | 245 | 202 | 208 |
| | 434 | 277 | 235 | 219 |
| Mean | 338.5714 | 250.2857 | 213.2857 | 199.1429 |
| S.D. | 120.0984 | 58.33728 | 64.33432 | 35.65309 |

1. A supplemented oil composition comprising therapeutically effective amounts of component being a marine oil concentrate and a second component being a compound selected from the group consisting of resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin, wherein
a. said composition is in a dosage form suitable for oral administration;
b. said composition comprises from at least 30 wt % to about 75 wt % of w-3 long chain polyunsaturated fatty acids selected from DHA and EPA;
c. said therapeutically effective amount is for use in treating a condition selected from the group consisting of mild to moderate hypertriglyceridemia, diabetes, atherosclerosis, mental cognition, metabolic syndrome, inflammation, and inflammation related disorders; and
d. the DHA:EPA ratio is from 10:0.01 to 1:10.

2. The composition according to claim 1, wherein the dosage form provides from 250 mg to 1,250 mg of the marine oil concentrate.

3. The composition according to claim 1, wherein the composition modulates one or more cardiovascular risk factors selected from the group consisting of omega-3 index, total cholesterol, LDL-cholesterol (direct measurement), HDL-cholesterol, Apo B, ApoA-1, Hs-CRP, albumin, ALAT, Alk Phosphatase, bilirubin, INR, TSH, BUN/Creatinine, complete blood count (CBC), aldosterone, angiotensin 2, oxidized LDL, resolvins, and renin.

4. A method for treating a condition selected from the group consisting of mild to moderate hypertriglyceridemia, diabetes, atherosclerosis, mental cognition, metabolic syndrome, inflammation, and inflammation related disorders in a subject in need thereof, said method comprising administering a supplemented oil composition comprising therapeutically effective amounts of a first component being a marine oil concentrate and a second component being a compound selected from the group consisting resveratrol, silibinin, alpha-lipoic acid, pterostilbene, N-acetyl cysteine, taurine, CoQ10, rosemary, rosemary extract, birch bark extract, leuteolin, ginger, betulin, betulinic acid, ECGC, acacia, THIAA, RIAA, tyrosol, hydroxytyrosol, mixed tocopherols, niacin, tocotrienols, citrus flavonoids, nobiletin, tangeretin, eriocitrin, and curcumin, wherein
a. said composition is in a dosage form suitable for oral administration;
b. said composition comprises from at least 30 wt % to about 75 wt % of w-3 long chain polyunsaturated fatty acids comprising at least DHA and EPA;
c. the DHA:EPA ratio is from 10:0.01 to 1:10.

5. The method according to claim 4, wherein the dosage form provides from 250 mg 1,200 mg of the marine oil concentrate.

6. The method according to claim 4, wherein the composition modulates one or more cardiovascular risk factors selected from the group consisting of omega-3 index, total cholesterol, LDL-cholesterol (direct measurement), HDL-cholesterol, Apo B, ApoA-1, Hs-CRP, albumin, ALAT, Alk Phosphatase, bilirubin, INR, TSH, BUN/Creatinine, complete blood count (CBC), aldosterone, angiotensin 2, oxidized LDL, resolvins, and renin.

7. The method according to claim 4, wherein the daily dose of the marine oil concentrate is from 1 gm to 6 gm.
